(19) United States
(12) Patent Application Publication
(10) Pub. No.: US 2010/0114262 A1
(43) Pub. Date: May 6, 2010

(54) LIGHT THERAPY DEVICE

(75) Inventors: LARRY VERNE PEDERSON, Medicine Hat (CA); JOEL CLEMENT LEONARD DAVID, Medicine Hat (CA); DENNIS ROGER FENRICK, Medicine Hat (CA)

Correspondence Address: BENNETT JONES LLP;C/O MS ROSEANN CALDWELL, 4500 BANKERS HALL EAST, 855 - 2ND STREET, SW, CALGARY, AB, T2P 4K7, CA

(73) Assignee: THE LITEBOOK COMPANY LTD., Medicine Hat (CA)

(21) Appl. No.: 12/564,188

(22) Filed: Sep. 22, 2009

Related U.S. Application Data
(63) Continuation of application No. 10/677,488, filed on Oct. 3, 2003.
Application No. 10/677,488 is a continuation-in-part of application No. 10/980,272, filed on Nov. 4, 2004.
Application No. 10/980,272 is a continuation of application No. 09/710,782, filed on Nov. 13, 2000, now Pat. No. 6,875,225.
(60) Provisional application No. 60/481,397, filed on Sep. 18, 2003.

(30) Foreign Application Priority Data
Mar. 14, 2000 (CA) 2300569
Aug. 30, 2000 (CA) 2317319

(51) Int. Cl. A61N 5/06

(52) U.S. Cl. 607/88

(57) ABSTRACT

A light treatment device including an outer housing, a light emitting assembly in the housing and operable to emit light through an opening in the housing, the light emitting assembly including a plurality of LEDs capable of generating light of less than 2,500 lux at 12 inches.

### CROSS REFERENCE TO RELATED APPLICATIONS

This application is a continuation of U.S. Ser. No. 10/677,488, filed Oct. 3, 2003 (pending), which is a non provisional claiming priority to U.S. provisional application Ser. No. 60/481,397, filed Sep. 18, 2003.

### FIELD

The present description relates to an ocular light therapy device and, in particular, to an ocular light therapy device for treatment of light deficient disorders.

### BACKGROUND

There is much support for the use of light therapy to overcome light deficient disorders. It has been proven that treatments involving shining light directly towards a user's eyes will alleviate or cure light deficient disorders including Seasonal Affective Disorder (SAD), circadian sleep disorders and circadian disruptions associated with jet-lag, shift-work, PMS, eating disorders and bulimia. Light therapy has also been shown effective for fatigue management.

A few types of light therapy devices presently available. One type of device is large in size and floor or desk mountable. These devices include light sources of fluorescent tubes. Although they can be moved from one position to another, they are not generally portable. In addition, the light source is quite fragile.

A second kind of light therapy device is head mountable. These devices are formed as eyeglasses or visors. While they are portable, they are not generally accepted by users for use in public because of their odd appearance when worn on the head. Also recent research has indicated that these devices may not orient the light source efficiently with respect to the users eyes, wherein a light source appears to best be a broad beam of light approaching the eyes from an angle. This combined with safety concerns about eye damage, given the proximity of the light source to the eye, has limited the acceptance of head mountable treatment devices.

These prior devices, therefore, are of limited use for persons requiring a portable and discreet treatment device. In today's mobile society, a light therapy device is needed for use by, for example, the business traveler, shift workers and far north or south residents that is portable, effective and aesthetically appealing.

### SUMMARY

In accordance with one aspect of the present invention, a light treatment device includes: an outer housing including a opening; a light emitting assembly in the housing and operable to emit light through the opening in the housing, the light emitting assembly including a plurality of LEDs capable of generating less than 2,500 lux at 12 inches.

In accordance with another aspect of the present invention, a light treatment device includes: an outer housing including a support base to support the device on a support surface spaced from a user; a light emitting assembly in the housing and operable to emit light from the device, the light emitting assembly including a plurality of LEDs capable of generating light of less than 2,500 lux at 12 inches.

In accordance with another aspect of the present invention, a light treatment device includes: an outer housing; a light emitting assembly in the housing and operable to emit light from the device, the light emitting assembly including a plurality of LEDs capable of generating light at less than 2,500 lux at 12 inches and being selected to emit light including at least one maximum peak in the 400 to 600 nm range of the spectrum.

In accordance with another aspect of the present invention, a light treatment device includes: an outer housing; a light emitting assembly in the housing and operable to emit light from the device, the light emitting assembly including a plurality of LEDs capable of generating light at less than 2,500 lux at 12 inches and being selected to emit light including at least one maximum peak in the 400 to 600 nm range of the spectrum and the at least one maximum peak includes an energy greater than or equal to 0.025 watts/m².

In accordance with another aspect of the present invention, a light treatment device includes: an outer housing; a light emitting assembly in the housing and operable to emit light from the device, the light emitting assembly including a plurality of LEDs capable of generating light at less than 2,500 lux at 12 inches and being selected to emit light wherein of the total light energy emitted 25 to 40% thereof is in the wavelengths 446 to 477 nm.

In accordance with another broad aspect of the present invention, a method for ocular light treatment includes: providing a device including (i) an outer housing, and (ii) a light emitting assembly in the housing and operable to emit light from the device, the light emitting assembly including a plurality of LEDs capable of generating light of less than 2,500 lux at 12 inches; setting the device at least 12 inches from a user; and operating the device to emit light toward and shining into the user's eyes.

In accordance with another broad aspect of the present invention, a method for ocular light treatment includes: providing a device including (i) an outer housing including a support base to support the device on a support surface spaced from a user; and (ii) a light emitting assembly in the housing and operable to emit light from the device, the light emitting assembly including a plurality of LEDs capable of generating light of less than 2,500 lux at 12 inches; setting the device on the support surface spaced at least 12 inches from a user; and operating the device to emit light toward and shining into the user's eyes.

In accordance with another broad aspect of the present invention, a method for ocular light treatment includes: providing a device including (i) an outer housing, and (ii) a light emitting assembly in the housing and operable to emit light from the device, the light emitting assembly including a plurality of LEDs capable of generating light of less than 2,500 lux at 12 inches and being selected to emit light including at least one maximum peak in the 400 to 600 nm range of the spectrum; setting the device at least 12 inches from a user; and operating the device to emit light toward and shining into the user's eyes.

In accordance with another broad aspect of the present invention, a method for ocular light treatment includes: providing a device including (i) an outer housing, and (ii) a light emitting assembly in the housing and operable to emit light from the device, the light emitting assembly including a plurality of LEDs capable of generating light of less than 2,500 lux at 12 inches and being selected to emit light including at least one maximum peak in the 400 to 600 nm range of the spectrum and the at least one maximum peak includes an energy greater than or equal to 0.025 watts/m²; setting the device at least 12 inches from a user; and operating the device to emit light toward and shining into the user's eyes.

In accordance with another broad aspect of the present invention, a method for ocular light treatment includes: providing a device including (i) an outer housing, and (ii) a light emitting assembly in the housing and operable to emit light from the device, the light emitting assembly including a plurality of LEDs capable of generating light of less than 2,500 lux at 12 inches and being selected to emit light wherein of the total light energy emitted 25 to 40% thereof is in the wavelengths 446 to 477 nm; setting the device at least 12 inches from a user; and operating the device to emit light toward and shining into the user's eyes.

### BRIEF DESCRIPTION OF THE DRAWINGS

FIG. 1 is a front elevation view of a light therapy device. A portion of the device has been cut away to facilitate illustration of internal components.

FIG. 2 is a side elevation view of the light therapy device of FIG. 1 with the support leg folded against the housing.

FIG. 3 is a sectional view along line A-A of FIG. 1.

FIG. 4 is a graph showing a spectra analysis of light emitted by one embodiment of a light therapy device.

FIGS. 5A and 5B are front and side elevations, respectively, of a support for permitting mounting of a light therapy device in a useful position. The support of FIG. 5A has secured therein a light therapy device.

FIG. 6 is a perspective view of another light therapy device in a closed configuration.

FIG. 7 is a perspective view of the device of FIG. 6 in an open configuration, ready for use.

FIG. 8 is an exploded view of the device of FIG. 6.

FIG. 9 is a schematic view of a method of light therapy.

FIG. 10 is another schematic view of a method of light therapy.

### DETAILED DESCRIPTION

A portable and lightweight, hand-held ocular light therapy device for treatment or prevention of light deficient disorders. The device can be durable, offering some resistance to damage by normal transport. The device can use light emitting diodes (LEDs) as a source of light. LEDs offer a light source that is lightweight, small in size, simple, durable as well as energy efficient. The device may be useful in confined spaces, during travel and for in-flight use while being aesthetically acceptable.

An ocular light treatment device can include an outer housing including a opening; a light emitting assembly in the housing and operable to emit light through the opening in the housing, the light emitting assembly including a plurality of LEDs capable of generating less than 2,500 lux at 12 inches.

The housing can be formed to permit the device to be mounted on a support surface or stand in a spaced relation from a user. For example, the housing can include a support base on which the device can be set on a support surface, the housing can include a support leg for supporting the device in an upright configuration and/or the housing can include an electrical contact for electrical connection to a mounting device. The support base, if one is included, can be formed in a flatted configuration and or can be weighted relative to the remainder of the housing to permit setting the device in an upright configuration. Alternately or in addition, the support base can be formed to be engaged by a holder for supporting the housing on a support surface in an upright configuration. The device can be generally intended to be operated at a distance of about 12 or more inches from the user and positioned with the opening toward the user's eyes so that the light emitted therefrom can pass directly or indirectly to the user's eyes.

The LEDs can provide a light emitting assembly that can be light-weight and durable. In one embodiment, the LEDs can be arranged in a pattern over an area and the light emitting assembly can be selected to emit light from the LEDs directly towards the user's eyes.

The light emitting assembly can include a screen of transparent or translucent material positioned over the LEDs, for example, across the opening to seal the housing and to prevent access to the LEDs and other internal components. The screen can be formed of light diffusing sheet material to provide a more uniform emission of light and/or to adjust the lux or characteristics of the light. While LEDs do not emit any significant amount of ultraviolet radiation, the diffuser sheet material can include a UV filter, if desired.

The LEDs can be selected to emit light illuminances of less than 2,500 lux measured at 12 inches from the assembly. The light levels can be selected in this range to be effective using reasonable treatment durations, but can reduce visual glare and other side effects and to simplify the device such as by reducing the number or power of LEDs and, accordingly, the size, cost and weight of the device. Lower light levels can also reduce device power requirements, therefore, facilitating the use of battery power.

The light emitted by the light emitting assembly either as emitted by the LEDs or as adjusted by a screen over the LEDs, can be selected to have a peak in the blue to green wavelengths of 400 to 600 nm and in one embodiment 450 to 550 nm. The emitted light can be exclusively in the blue to green wavelengths such that it visually appears blue to green. Alternately, the light emitted can include a spectrum of wavelengths with a maximum peak in the range of 400 to 600 nm. Such light may, for example, appear as white light. While there may be more than one peak wavelength in the emitted light, the major peaks are preferably in the 400 to 600 nm range. In one embodiment, a maximum peak wavelength can in the blue region of the spectrum, which is 420 to 505 nm. In one embodiment, a maximum peak wavelength can be in the range of 446 to 477 nm.

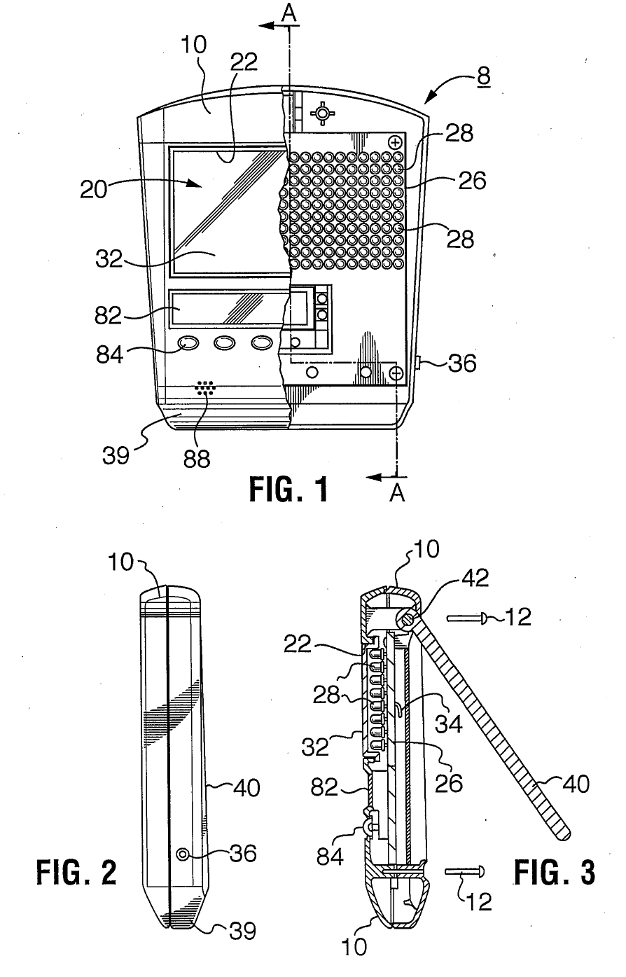

Referring to FIGS. 1 to 3, a light therapy device 8 according to one embodiment is shown. The device can be small in size, for example, resembling a large calculator or hand-held computer. The outside dimensions of the device can be less than about 7 inches×7 inches×1.5 inches. The size can be varied as desired and with consideration as to portability, convenience and the components that must be contained within the device.

The device can include an outer housing 10. The housing can be formed of a durable, impact resistant material such as, for example, a polymer (i.e. nylon, thermoplastics or blends thereof). All housing parts can be of minimal thickness to provide suitable impact resistance and support for internal components while minimizing the weight of the device. The housing can be formed in various ways, for example, from injection molded parts secured together by screws 12 or other fasteners, polymeric welding, fusing, adhesives, etc.

The housing can carry a light emitting assembly 20. The light emitting assembly can be mounted in the housing such that, during operation, light emitted therefrom is directed out through an opening 22 in the housing. The light can be emitted in a broad, as opposed to a focused, beam. The broad beam can increase in its width with increasing distance from the device so that light impinging on the user is about shoulder width (30 to 50 inches). For example, in one embodiment, light can be emitted from the device at an angle of about 10° to 30° from an axis oriented orthogonally through the plane of the opening. In one embodiment, the light emitting assembly can generate a beam of light that radiates out through the opening having a width of about 4.5″ to a beam width of about 40″ at 24″ from the device. This then can create a treatment field of about shoulder width when the device is operated at 24″ from the user.

Light emitting assembly 20 can include a printed circuit (PC) board 26 providing electrical connection for light emitting diodes 28. The LEDs can be mounted in various ways, for example as by traditional mounting or surface mounting. A screen 32 can be mounted over the light emitting diodes and across the housing opening to prevent access to the internal components of the device. If a screen is used, it is useful to ensure that appropriate light characteristics, as set out herein, can passed therethrough to permit treatment.

The LEDs can be spaced apart on board 26, with consideration as to their light output and emission wavelength, such that the assembly emits a light illuminance adequate for treatment of a light deficient disorder. In particular, the light emitting assembly can generate adequate illumination for treatment of light deficient disorders including Seasonal Affective Disorder (SAD), circadian sleep disorders, fatigue and circadian disruptions associated with jet-lag, shift-work, PMS and eating disorders such as binging, cravings and bulimia. These illuminances can be less than 2,500 lux at 12 inches from the assembly. To generate this level of illumination, the assembly generally can include between about 10 and 150 LEDs. Depending on the output of the LEDs, in one embodiment, 24 to 72 LEDs can be used in a device and in another embodiment, 36 to 60 LEDs can be used.

To reduce treatment duration regimens, light at a level of less than 2,500 lux can have optimized wavelength emissions with peaks ranging between 400 to 600 nm. In one embodiment, a device emits light with peaks in the 450 to 550 nm range. In another embodiment, a peak wavelength can be in the blue region of the spectrum, which is 420 to 505 nm. Using a light therapy device with light illuminances of less than 2,500 lux and wavelengths peaked in the blue to green region of the spectrum, treatments of acceptable duration can be administered. As an example, treatments for SAD can be completed in ¼ to 4 hours and in most cases, ½ to 3 hours.

The light generated by the device can be predominantly in the blue to green region such that the emitted light appears distinctly blue/green to a user. However, to enhance acceptance and to reduce the occurrence of problematic after-images, the light can include a range of wavelengths such that the emitted light appears white, but can include a maximum peak in the 400 to 600 nm range.

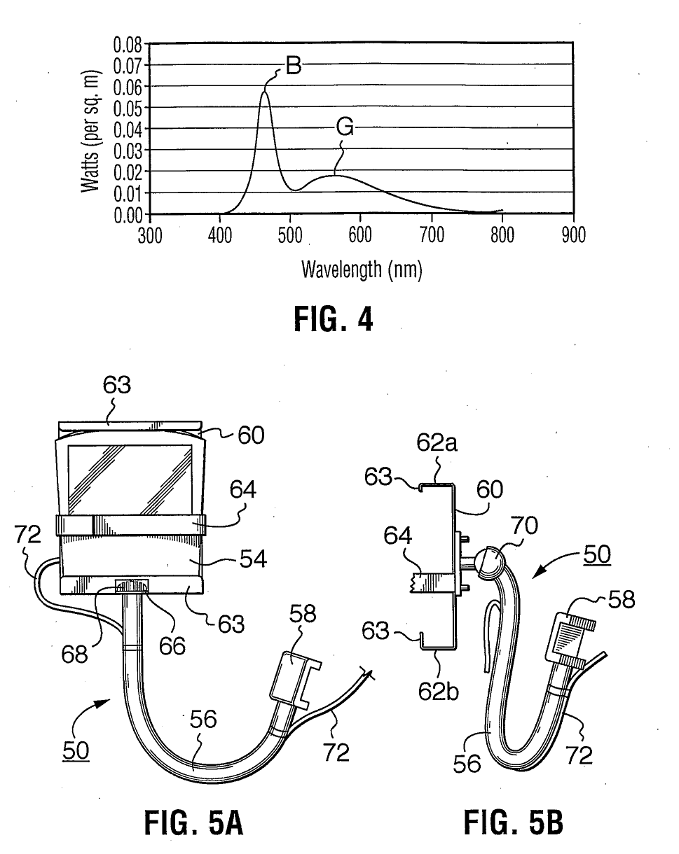

FIG. 4 shows a spectra analysis of light generated by a light therapy device at 12 inches. The light appears as a bright white light, but can have a maximum peak B in the blue wavelengths, between about 446 nm and 477 nm with peak B centered at about 464 nm and with an energy of about 0.055 watts/m². The light emission further can include a secondary but significant peak G in the green wavelengths, between about 505 nm to 600 nm with the greatest output in this peak at about 555 nm. Light emitted can have a maximum peak wavelength in the relevant wavelengths with an energy greater than or equal to 0.01 watts/m². In another embodiment, the emitted light can have a maximum peak has an energy greater than or equal to 0.025 watts/m².

In one embodiment, a light therapy device can emit light wherein of the total light energy emitted at least 25% thereof is of the wavelengths 446 to 477 nm. In another embodiment of a light therapy device, the total light energy emitted is 25 to 40% in the wavelengths 446 to 477 nm.

To achieve a light emission of less than 2,500 lux with peak emissions in the 400 to 600 nm region of the spectrum, various approaches can be taken. In one embodiment, a screen can be used that filters out all or a portion of the less desirable wavelengths. In another embodiment, LEDs capable of emitting only selected wavelengths, for example, including blue, yellow and green, can be used. In yet another embodiment, white light LEDs having selected peak wavelengths can be used.

Power can be supplied to the LEDs through electrical lines 34. Power can be provided through batteries or, to reduce weight, through a jack 36 for connection to an electrical supply (for use in North America). The device can operate using DC power and is supplied with an external AC-DC converter. Since the device can be useful during long distance travel in the treatment of jet lag, an adapter can be provided within the device or separately for device compatibility with foreign voltages of AC power or with DC power, as is provided through power ports mounted in aircraft armrests.

Device 8 of FIG. 1 can be of hand-held configuration, portable and selected to be set or mounted on a support surface and spaced a distance of about 12 or more inches from the user for use. As such, the housing can include a support base 39 on which the device is set for use. To facilitate light treatment, a support leg 40 can be provided for supporting the housing in a propped position such that light can be emitted in a generally upward (such as 30 to 60° from horizontal) direction from its supported position. In one embodiment, support leg 40 can be connected by a hinge 42 to the rear of the housing such that the leg can be rotated between a supporting position and a stored position against the rear of the housing. Another stand for supporting or elevating the light illuminating assembly can be used, as desired.

The light treatment device can be mounted for use in other ways. For example, a device can be permanently or removably mounted in a vehicle passenger compartment including, for example, a passenger or operator seat area or a sleeper unit of a transport truck. The vehicle can be, for example, an aircraft, a train, a bus, a truck, a boat or an automobile. In one embodiment, the light treatment device can be mounted or mountable in an aircraft seat back or in an aircraft seat armrest for use by air travelers. The device can be mounted in a manner similar to aircraft telephones, individual video monitors, and other such devices, wherein the light treatment device can be attached to an adjustable extension arm, thereby enabling the user to remove the light treatment device from an armrest and position it appropriately for treatment. Alternately, the light treatment device may be temporarily removed from its seat back mounting position and positioned on a tray table or other support surface for treatment, while remaining secured to the seat back by means of a cable that could also serve as a device connection to a power source. The device may also be mounted into an airliner flight deck, crew rest area or other such areas of an airliner to provide discreet and convenient light treatments for pilots, flight attendants and other such on-board crew affected by jet lag and fatigue.

In another embodiment, the light treatment device can be mounted in the passenger compartments of vehicles, for example, automobiles, transport trucks, buses, trains, and other such vehicles, wherein the device is stored when not in use but readily available to provide a light therapy treatment. In the case of automobiles and trucks, the device may be mounted on the underside of a sun visor, or within the glove compartment, under the vehicle's dashboard, in the back seat or in the sleeper compartment. The device can be attached to an adjustable extension arm in order to permit proper positioning for treatment.

In another embodiment, a device can be mounted to permit use for example on a wheel chair or a hospital or other bed. In yet another embodiment, the device can be mounted to permit a light treatment while operating a fitness machine, such as a stationary bicycle, a treadmill, elliptical trainer or a stair climbing simulator.

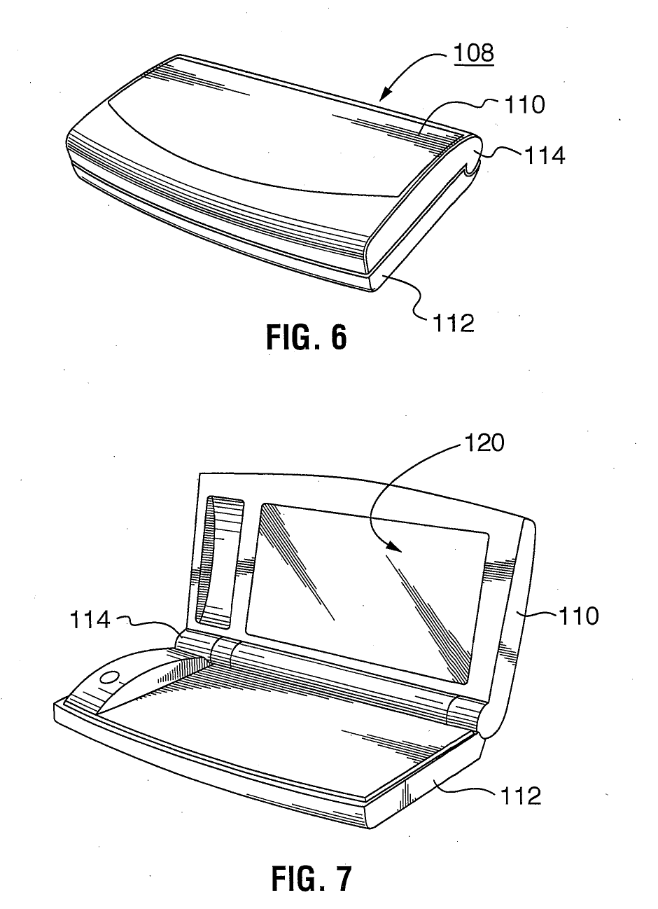

Referring to FIG. 5, a support 50 can be useful for mounting a light treatment device 54. Support 50 can include a bendable arm 56 of the type including a corrugated tube and internal supports that can be bent into various orientations and, once positioned, will hold fast in that orientation. A clamp 58 or other securing device can be mounted at a first end of arm 56 for securing the arm to a support surface such as a member of a wheel chair, fitness machine, etc. At the opposite end of the arm can be mounted a frame 60 into which a light therapy device can be secured.

Frame 60 can include an upper and lower walls 62a, 62b with returns 63 and a fastening strap 64. Device 54 can be slid between walls 62a, 62b and held in place by returns 63 and strap 64. Lower wall 62b can include an opening 66 such that the device can be positioned without its vents 68 blocked.

Frame 60 can be connected to arm 56 using a ball joint 70, which permits adjustment of the support relative to the arm.

Arm 56 and ball joint 70 can be adjustable yet rigid enough to hold the weight of light treatment device 54 and frame 58 without moving out of the bended configuration into which it has been oriented.

A power cord 72 can be connected to extend with arm 56 to provide electrical communication to the device. The power cables can be housed within arm 56 if desired.

As such, support 50 can support a device for a light treatment. However, the device can be easily removed from the adapter for carrying to another location. Of course, other adapters can be used, for example, ones having rigid arms or supports offering more permanent securing of the device.

Housing 10 can be formed in other ways, for example, for other applications, to accommodate other electronics, batteries etc. or to define storage space such as for cords, adapters, glasses or other items. The housing can also include a cover or a case.

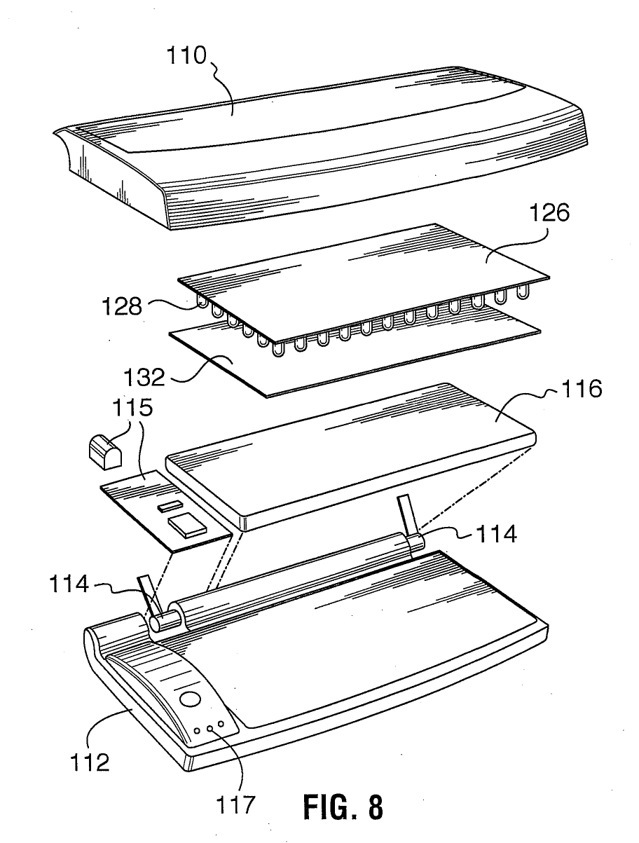

Referring to FIGS. 6 to 8, a light therapy device 108 according to another embodiment is shown. The device can have an outer housing including an upper housing member 110 and a lower housing member 112. The housing members can be connected by a hinge 114 that permits them to pivot relative to each other between a closed position shown in FIG. 6 and an open position shown in FIG. 7. When in the closed position, the housing members can be locked together by resistance in the hinge. The device can be small in size and, when closed, can resemble a portable compact disc player or a make-up compact.

The housing can enclose a light emitting assembly 120 including a PC board 126 with LEDs 128 mounted thereon and a screen 132. In the illustrated embodiment, light-emitting assembly 120 can be mounted in the upper housing member. The light emitting assembly can be mounted on the inwardly facing portion of the upper housing member so that, when the device is in the closed position, assembly 120 can be protected within the housing members. In this way, the light emitting assembly, which is more fragile than the housing, can be protected against damage during transport.

The device can be opened for use to administer a light treatment. In a one embodiment, upper housing member 110 can be unfolded from the closed position by rotating about hinge 114. Lower housing member 112 can act as a base for supporting the light emitting assembly. Hinge 114 can be of the type that permits self-locking in at least a few rotational orientations. The use of such a hinge permits that, for example, upper housing member can be oriented to direct the light in a plurality of directions. This is useful as it may be necessary, depending on the support surface on which the device is set or the height of the user, to adjust the direction of the emitted light.

Counterweights (not shown) can be mounted in the lower housing member to prevent the device from tipping. Member 112 can accommodate electronics including, for example, boards or power or communication jacks, indicated generally at 115, batteries 116 or formed to define storage space such as for cords, adapters, glasses or other items.

Device 108 can operate on rechargeable batteries and can include indicator lights 117 including those indicating operation and battery status.

Device 108 can accommodate a processor at 115 to calculate a light treatment regime based on installed programs or input of information. A communication hardware and software can be provided for download of information from external sources such as from the Internet. The processor can include a feature that turns the device on at a pre-set time for a specific duration. A touch screen control option can be incorporated to facilitate use.

Device 8, of FIGS. 1 to 3, can accommodate a calculator including a display 82, a key pad 84, and a processor. The calculator can be programmed to calculate a light treatment regime based on input of information. The calculator processor can use calculation references such as that known as the Jet Lag Calculator™ available from Bio-Brite, Inc., Maryland or other sources. In one embodiment, the calculator can be used to calculate light treatment regimes for jet lag based on inputs of information, as follows:

Option 1
i. Number of time zones crossed during trip
ii. Direction of time zones crossed (East or West)
iii. Normal wake-up time of user (for establishing the user's “body clock”)

Option 2
i. Departure city
ii. Arrival city
iii. Normal wake-up time of user

Based on the input of the above-noted information, the calculator can then calculate and display a treatment regime including, for example, a period of light exposure and a period of light avoidance. In option 2, the calculator can determine the number of time zones through which travel will occur and can use this to calculate treatment regime. The calculator in one embodiment can calculate a two-day treatment regime.

In one embodiment, the calculator keypad can include keys to be depressed when inputting particular information. As an example, the keypad can include keys such as: “departure city”, “destination city” and “wake up time”. The calculator can be adapted to prompt the user such as by displaying questions requesting the appropriate information. The calculator can include a pause function capable of recording a time of treatment interruption and capable of outputting from memory the portion of the treatment remaining when treatment is resumed.

In addition or alternately, the calculator can be programmed for calculation of other treatment regimes such as, for example, for treatments to alleviate fatigue in shift workers or long-haul trucking or transport (i.e. truck drivers, train engineers or bus drivers). Treatments for shift workers may include inputs such as work shift start time, previous shift time and normal waking time.

A speaker 88 can be provided for communication to the user. As an example, the speaker can be driven by the calculator processor to audibly prompt a user to input information. In addition, the speaker can function to emit an audible signal, such as an alarm, to alert a user to commence or modify a treatment. In one embodiment, the calculator processor can control a switch for the light emitting assembly such that it can be turned on or off in response to a signal from the processor.

In one embodiment, the calculator memory can store previous treatment regimes. These stored treatment regimes can be recalled from processor memory for repeat trips or shift work schedules.

If desired, to enhance the usefulness of the device, the calculator can also be programmed with other information including a clock, a standard mathematical calculator or other information such as an address book, etc.

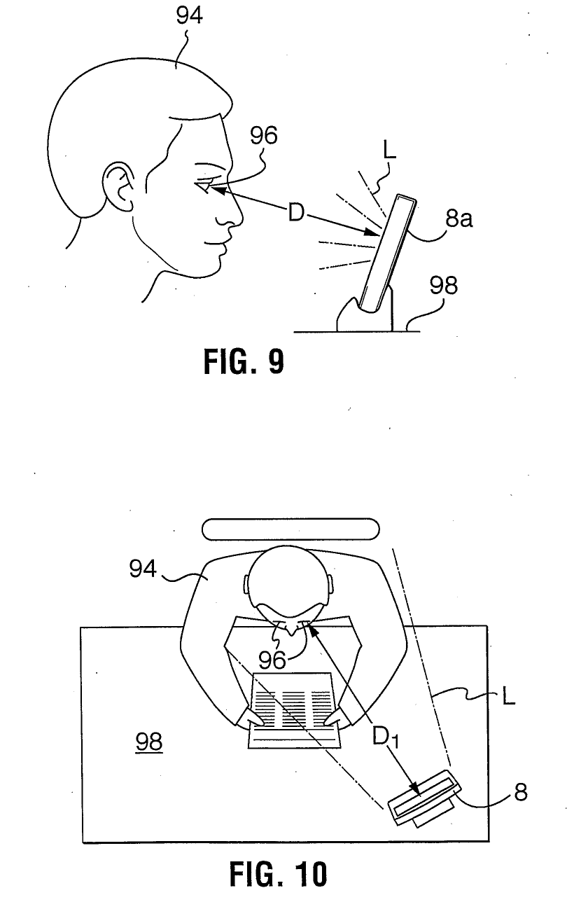

Referring to FIGS. 9 and 10, a method for light therapy can include spacing a light therapy device 8, 8a a distance D, D₁ of 12 or more inches from a user 94, who can be suffering from a light deficient disorder, but can alternately be, for example, seeking to avoid a light deficient disorder or adjusting their body clock due to jet lag or shift work. The device can then be operated to emit light L at levels of less than 2,500 lux and possibly, as discussed hereinabove, with a maximum peak emission in the 400 to 600 nm region of the spectrum and directing the light toward the eyes 96 of the user. To effect treatment, the light emitting assembly can be directed toward the user, with the emitted light from the device shining into the user's eyes. The present device can be used to provide ocular treatment for all applications and indications and therefore can be used while the users eyes remain substantially open, rather than while they are sleeping.

Typically, the user can position the light emitting assembly of the device between 12-24 inches from their eyes so that a broad beam of light, about shoulder width, impinges on the user. The treatment field generated by the device can offer personal light therapy. Since the treatment field at normal spacings can be shoulder width, the device can be used without shining the emitted light onto adjacent persons.

The device can be situated on a support surface 98 such as a table, desk, etc. or supported in other ways such as by the assembly of FIG. 5, so as to emit light upwards towards the user's eyes. The device can be offset, for example, 30 to 45° (FIG. 10), from a position directly in front of the user, so that the light shines directly on the periphery of the retina (outside the fovea), which is thought to be the location of the photoreceptors responsible for shifting of the human body clock.

The user's eyes should be open to effect treatment, although blinking to a normal degree is expected and permitted. It is not necessary for the user to stare directly into the light from the device. Indeed, the light is generally sufficiently bright so that the user instinctively knows not to do so.

Treatment times for SAD are typically 15-60 minutes/day. Previous studies have shown the most effective light treatment times to be as soon as possible upon waking each morning during the ‘seasonal’ period for S.A.D. (in the northern hemisphere including North America, northern Europe, etc., the SAD season is September-March annually).

Treatment times to ‘seek light’ for ‘jet-lag’ are typically a 3-hour period on the day of arrival (Day 1) in the new destination, as determined by the inputs for departure city, arrival city, and normal waking time. A similar period can be recommended to ‘avoid light’, wherein the user wears light-blocking glasses if outdoors in direct sunlight. Some previous studies have shown that a substantially shorter light therapy treatment period has effected the desired benefit; in some cases, as short as 45 minutes on Day 1 of travel, and 15 minutes on Day 2 effected a complete 6 time zone shift.

Treatment regimens for additional applications, e.g. PMS, Delayed Sleep Phase Syndrome, etc. typically indicate a treatment regimen of similar duration as for SAD, except that in some applications (i.e. Advanced Phase Sleep Syndrome) treatment occurs in the evening (to delay the onset of melatonin secretion).

Treatment for fatigue management may include short light treatments using the light device, during or following a rest period and prior to initiating work or normal activities.

Numerous modifications, variations and adaptations may be made to the particular embodiments described above without departing from the scope of the invention as defined in the claims.

What is claimed is:

1. An ocular light treatment device comprising: an outer housing including an opening; a light emitting assembly in the housing and operable to emit light through the opening in the housing, the light emitting assembly including an LED light source capable of generating an output of light suitable for ocular light therapy and of less than 2,500 lux at 12 inches.

2. The ocular light treatment device of claim 1 wherein the light emitting assembly is selected to emit light including a maximum peak in the 400 to 600 nm range of the spectrum.

3. The ocular light treatment device of claim 2 wherein the emitted light includes a range of wavelengths such that the emitted light appears white.

4. The ocular light treatment device of claim 2 wherein the maximum peak includes an energy greater than or equal to 0.010 watts/m².

5. The ocular light treatment device of claim 2 wherein the maximum peak includes an energy greater than or equal to 0.025 watts/m².

6. The ocular light treatment device of claim 1 wherein the light emitting assembly is selected to emit light including a maximum peak between about 420 nm and 505 nm.

7. The ocular light treatment device of claim 5 wherein the maximum peak includes an energy greater than or equal to 0.010 watts/m².

8. The ocular light treatment device of claim 1 wherein the light emitting assembly is selected to emit light wherein of the total light energy emitted at least 25% thereof is of the wavelengths 446 to 477 nm.

9. The ocular light treatment device of claim 7 wherein of the total light energy emitted 25 to 40% thereof is in the wavelengths 446 to 477 nm.

10. The ocular light treatment device of claim 1 wherein the light emitting assembly is selected to emit light including a maximum peak between about 505 nm to 600 nm.

11. The ocular light treatment device of claim 9 wherein the maximum peak includes an energy greater than or equal to 0.010 watts/m².

12. The ocular light treatment device of claim 1 wherein the light emitting assembly includes a screen selected to filter the emitted light such that the emitted light includes a maximum peak in the 400 to 600 nm range of the spectrum.

13. The ocular light treatment device of claim 1 wherein the LEDs are capable of emitting light peaked in the 400 to 600 nm range of the spectrum.

14. The ocular light treatment device of claim 12 wherein the LEDs are white light emitting.

15. The ocular light treatment device of claim 1 wherein the light emitting assembly is selected to emit light in a beam that has a width increasing with distance from the device.

16. The ocular light treatment device of claim 14 wherein light is emitted from the device at an angle of about 10° to 30° from an axis oriented orthogonally through a plane defined by the opening.

17. The ocular light treatment device of claim 1 further comprising a support base to support the device on a support surface spaced from a user.

18. The ocular light treatment device of claim 16 wherein the support base is detachable from the housing.

19. The ocular light treatment device of claim 16 wherein the support base is connected to the housing.

20. The ocular light treatment device of claim 16 wherein the support base is integral to the housing.

21. A method for ocular light treatment comprising: providing a device including (i) an outer housing, and (ii) a light emitting assembly in the housing and operable to emit light from the device, the light emitting assembly including a plurality of LEDs capable of generating capable of generating an output of light suitable for ocular light therapy and of less than 2,500 lux at 12 inches; setting the device at least 12 inches from a user; and operating the device to emit light toward and shining into the user's eyes.

22. The method for ocular light treatment of claim 21 wherein the emitted light includes a maximum peak in the 400 to 600 nm range of the spectrum.

23. The method for ocular light treatment of claim 21 wherein the emitted light includes a maximum peak between about 420 nm and 505 nm.

24. The method for ocular light treatment of claim 21 wherein of the total light energy emitted at least 25% thereof is of the wavelengths 446 to 477 nm.

25. The method for ocular light treatment of claim 21 wherein the emitted light is selected to emit light including a maximum peak between about 505 nm to 600 nm.

26. The method for ocular light treatment of claim 21 wherein the light emitting assembly is selected to emit light in a beam that has a width increasing with distance from the device.

27. The method for ocular light treatment of claim 21 wherein in the step of operating the device to emit light toward and shining into the user's eyes, the light impinging on the user is in a beam of about 30 to 50 inches horizontal width.

28. The method for ocular light treatment of claim 21 wherein the emitted light is directed upwardly toward the user's eyes.

29. The method for ocular light treatment of claim 21 wherein the device is set on a support surface.
